(19) United States
(12) Patent Application Publication
(10) Pub. No.: US 2026/0283173 A1
(43) Pub. Date: Sep. 24, 2026

(54) UV DISINFECTOR FOR OPTICALLY DENSE FLUIDS

(71) Applicant: Ultra Biotecs Limited, Northampton (GB)

(72) Inventor: Malcolm Snowball, Hawkhurst Kent (GB)

(21) Appl. No.: 19/474,433

(22) PCT Filed: Apr. 11, 2024
(86) PCT No.: PCT/GB2024/050952

§ 371 Date: Oct. 10, 2025

(30) Foreign Application Priority Data
Apr. 13, 2023 (GB) 2305432.3

(51) Int. Cl. A23B 70/50; A23B 2/00; A23B 2/53; B01F 23/45; B01F 25/421; B01F 101/00; B01F 101/14; B08B 9/023

(52) U.S. Cl. CPC A23B 70/50; A23B 2/001; A23B 2/53; B01F 23/45; B01F 25/4233; B08B 9/023; B01F 2101/14; B01F 2101/2204; B08B 2209/02

(57) ABSTRACT

The disclosure provides apparatus for disinfection of optically dense fluid including a metal tube, a quartz tube surrounded by the metal tube, a UV lamp surrounded by the quartz tube and a chamber. The chamber is defined between the quartz tube and the metal tube and has an inlet and an outlet. A cleaning sleeve is positioned in the chamber and is movable along the quartz tube between a start position and a stop position. A valve arrangement transfers optically dense fluid to spaces behind and in front of the cleaning sleeve to move the cleaning sleeve between its start position and its stop position. Also disclosed is a mixer for mixing fluid and a method for disinfection of optically dense fluids.

### FIELD OF THE INVENTION

This invention relates to a method and apparatus for the disinfection of optically dense fluids such as concentrates and in particular but not solely to drinks and drink products.

### BACKGROUND

Previous attempts to use thin film UV disinfection techniques together with mixers have not been successful because of an inability to keep the UV lamp operating on top efficiency and an inability to create highly efficient mixing of the fluid.

The inventor has come to the conclusion that the causes of these major problems are:
1) Proteins in the liquids adhere to the quartz tube surrounding the UV lamp. Proteins naturally have a high affinity to quartz and will readily bind to the quartz and they are also total absorbers of the 254 nm UV light. In effect the UV light is significantly diminished over time and the UV disinfector is unable to disinfect the liquid. This coating is very difficult to remove by conventional clean in place processes used in the drinks industry.
2) There is usually a lot of debris in a dense liquid providing many places for microorganisms to be shielded from the UV light therefore it is imperative that the mixing technique is consistent and very efficient and that all of the liquid is exposed to the UV light.

The following invention aims to solve the aforementioned problems.

### SUMMARY OF THE INVENTION

Aspects of the invention are set out in the independent claims and optional features are set out in the dependent claims. Aspects of the invention may be provided in conjunction with each other and features of one aspect may be applied to other aspects.

An aspect provides apparatus for disinfection of optically dense fluid, the apparatus comprising any of all of the following features: a metal tube; a quartz tube surrounded by the metal tube; a UV lamp surrounded by the quartz tube; a chamber defined between the quartz tube and the metal tube, the chamber having an inlet and an outlet; a cleaning sleeve positioned in the chamber and configured to be movable along the quartz tube between a start position and a stop position; and a valve arrangement configured to transfer optically dense fluid to spaces behind and in front of the cleaning sleeve to thereby move the cleaning sleeve between its start position and its stop position.

The apparatus may address the problem of the build up of a coating on the quartz tube by providing a cleaning sleeve and a valve arrangement which can automatically clean the quartz tube, thereby avoiding the need for manual cleaning (e.g., by hand).

The UV lamp may be configured to provide UV light having a wavelength in the range from 250 nm to 270 nm. The wavelength may be 254 nm.

The apparatus may comprise a flange rigidly fixed to each end of the metal tube. The flanges may comprise a clearance hole to allow the quartz tube to protrude through the flanges so that the flanges are positioned centrally with the metal tube.

The metal tube may be made of stainless steel. An inside surface of the metal tube may be coated with polytetrafluoroethylene, PTFE.

The cleaning sleeve may comprise two end flanges. The two end flanges may be interposed by a knitted or compressed wire sleeve.

An inner diameter of the metal tube may be approximately 5 mm greater than an outer diameter of the quartz tube.

At least one end of the cleaning sleeve may comprise a pin configured to limit the travel of the cleaning sleeve.

The inlet and the outlet of the chamber may be positioned such that fluid communication with the chamber is maintained when the cleaning sleeve is at its start position and its stop position.

The apparatus may further comprise at least one of: a bleed pipe configured to provide a conduit between the outlet and the space behind the cleaning sleeve; and a bleed pipe configured to provide a conduit between the inlet and the space in front of the cleaning sleeve.

The valve arrangement may comprise: a forward valve configured to feed optically dense fluid into the space behind the cleaning sleeve via a forward manifold; a return valve configured to feed optically dense fluid into the space in front of the cleaning sleeve via a return manifold; and a pump configured to pump optically dense fluid from the outlet of the chamber to the forward valve and the return valve.

The apparatus may further comprise a mixer. The mixer may be for chaotically mixing the optically dense fluid between the inlet and the outlet.

The cleaning sleeve may be a first cleaning sleeve and the apparatus may further comprise a second cleaning sleeve. The second cleaning sleeve may be positioned in the chamber and configured to be movable along the quartz tube between a start position and a stop position.

The first cleaning sleeve may be disposed in a first chamber segment. The second cleaning sleeve may be disposed in a second chamber segment. The second chamber segment may be fluidically separated from the first chamber segment, for example by a flange.

The valve arrangement may be further configured to transfer optically dense fluid to spaces behind and in front of the second cleaning sleeve to thereby move the second cleaning sleeve between its start position and its stop position.

An aspect of provides a mixer for mixing fluid, the mixer comprising any or all of the following features: a cylindrical body; an inlet pipe for transporting fluid into the cylindrical body; an outlet pipe for transporting fluid out of the cylindrical body; a central support shaft; and a plurality of mixing discs rigidly attached to the central support shaft via a central mounting hole; wherein each of the plurality of mixing discs comprises an array of holes on one side of the mixing disc; and wherein the mixing discs are arranged such that pressurised fluid entering the inlet pipe is forced through the array of holes of each of the plurality of mixing discs to provide chaotic mixing of the fluid before reaching the outlet pipe.

The plurality of mixing discs may comprise: a first mixing disc comprising a first array of holes; and a second mixing disc, adjacent to the first mixing disc, and comprising a second array of holes; wherein the first array of holes is on the opposite side to the second array of holes.

The inlet pipe may be provided at a base of the cylindrical body. The inlet pipe may be offset from the central support shaft by a first radial distance. At least one of the plurality of mixing discs may be arranged such that its array of holes is provided at the first radial distance.

An aspect provides a method for disinfection of optically dense fluid comprising any or all of the following steps: directing fluid to a chamber, the chamber being defined between a metal tube and a quartz tube that surrounds a UV lamp; irradiating the fluid with UV light from the UV lamp; directing the fluid from the chamber to a valve arrangement, the valve arrangement configured to direct fluid to spaces behind and in front of a cleaning sleeve surrounding the quartz tube in the chamber; and scrubbing the quartz tube with the cleaning sleeve, wherein scrubbing comprises: opening a first valve of the valve arrangement to direct fluid to the space behind the cleaning sleeve to move the cleaning sleeve forwards; and closing the first valve and opening a second valve of the valve arrangement to direct fluid to the space in front of the cleaning sleeve to move the cleaning sleeve backwards.

The step of directing fluid to the space behind or in front of the cleaning sleeve may be performed at a higher pressure than the step of directing fluid to the chamber. The pressure of the liquid delivered to the space behind or in front of the cleaning sleeve may be in the range from 1 to 5 bar.

The invention provides highly effective automatic cleaning systems together with very high efficiency mixers based on chaotic mixing techniques.

Aspects may also have the following features.

A UV lamp is surrounded centrally by a quartz tube which is in turn surrounded centrally by a metal tube. Rigidly fixed to each end of the metal tube is a flange. The flanges have a clearance hole to allow the quartz tube to protrude through the flanges and are positioned so that they are central with the metal tube.

Preferably the metal tube is made of a food grade stainless steel.

Preferably the inside surface of the stainless steel tube is coated with PTFE.

Preferably provision is made for an automatic cleaner.

Positioned centrally around the quartz tube inside the metal tube is a cleaning sleeve consisting of two end flanges interposed with a knitted or compressed wire sleeve.

Preferably the end flanges are made of PTFE.

Preferably the end flanges are a small clearance fit between the outside diameter of the quartz and the inside diameter of the metal tube.

Preferably the cleaning sleeve is made from stainless steel wire.

Preferably the cleaning sleeve is made as an interference fit between the outside diameter of the quartz tube and the inside diameter of the stainless steel tube. When manufactured the cleaning sleeve acts like a circumferential tubular spring lightly gripping the outside diameter of the quartz tube and lightly compressing against the inside surface of the metal tube. Attached to one end of the cleaning sleeve is a pin which acts as a stop and positions the cleaning sleeve at its start point in the cleaning cycle. Attached to the other end of the cleaning sleeve is another pin which acts as a stop which positions the cleaning sleeve at its finish point in the cleaning cycle. This subassembly constitutes one segment. The segment has an inlet and an outlet positioned such that the cleaning sleeve does not impinge on either the inlet or the outlet.

The last segment's outlet has a flow through on/off valve which feeds into the UV disinfector's output manifold so that the UV disinfector's flow can be isolated.

Several of these segments can be fixed together with the quartz tube containing the UV lamp running through the segments to form the UV disinfector.

The segment is made watertight with an “O” ring which when the segments are clamped together the “O” ring is compressed between the outside surface of the quartz tube and the flange. The first and last segments have clamping rings fitted to form the “O” ring seal.

Straddling the segments watertight joints are mixers to mix the fluid; any fluid flowing through the disinfector is forced to go through the mixer therefore avoiding any chance of any part of the fluid bypassing the mixing process and therefore ensuring that all of the fluid is mixed. Chaotic mixing of the liquid may be achieved by pressurising the fluid at the input to the mixer at a pressure in the range from 1 to 2 bar.

The mixer comprises a cylindrical body which is domed at one end and flanged at the other end. The cylindrical body is clamped to a base which has a machined “O” ring groove and “O” ring together with clamping holes so when the cylindrical body is clamped to the base it forms a watertight joint. The base has a rigidly fixed inlet pipe so that fluid can enter the cylindrical body.

Centrally attached to the base is the mixing apparatus complete with “O” ring to ensure a watertight seal between the base and the mixing apparatus. The mixing apparatus is designed to produce chaotic mixing of all of the fluid.

If a jet of water is sprayed directly onto a solid wall the water rebounds off the wall in all different directions and at the same time the water molecules in the jet are projected into other water molecules and cause them to scatter in totally random directions; this is chaotic mixing and is a very efficient way of mixing fluids.

The mixing apparatus consists of a central support shaft. Means are provided to fix the support shaft to the base in the form of a screw thread at the base of the support shaft. Rigidly attached to the support shaft are mixing discs spaced equally apart. Each disc has a grooved “O” ring attached to its outer rim sized such that when the cylindrical body is attached it provides a watertight seal between the disc and the inside surface of the cylindrical body. The discs are thin metal plates with an array of holes at one side of each disc and a central mounting hole. The discs are rigidly fixed to the support shaft; the first disc is attached with the array of holes on the same centre line as the inlet pipe but at the opposite side of the disc. All of the other discs are fixed equally spaced with their array of holes placed at the opposite side to the previous disc and in line with the previous discs array of holes.

When a fluid under pressure enters the mixer from the inlet pipe it immediately hits the base of the first disc where the water molecules scatter in a random pattern providing chaotic mixing. Due to the pressure the fluid is forced through the array of holes at the other end of the disc producing jets of fluid which hit the base of the second disc and creates further chaotic mixing. This effect carries on as the fluid progresses through the mixer.

Means are provided to deliver the mixed fluid out of the mixer and into the UV disinfector in the form of an outlet pipe rigidly fixed to the centre of the domed top of the cylindrical body. Both the inlet and the outlet have a flange rigidly attached; both flanges have an “O” ring groove such that when the flanges are clamped with an “O” ring onto the outlet and inlet of the segments they form a watertight seal.

Preferably the mixer is made from food grade stainless steel.

Means are provided to move the cleaner from its start position to its stop position and from its stop position to its start position in the form of twin on/off flow through valves. One valve feeds into a forward manifold and the other valve feeds into a return manifold. The forward manifold feeds into the space behind the cleaning sleeve when it is in its start position and the return manifold feeds into the space in front the cleaning sleeve when it is in its stop position. Feeding the two valves is the cleaning manifold which in turn is fed from a pump whose inlet is fed from the UV disinfector's outlet manifold. The pump draws fluid from the UV disinfector's output manifold and pressurizes the cleaner manifold with a pressure higher than the inlet pressure and with the on/off valve shut and the forward valve open moves the cleaning sleeve to its stop position. With the on/off valve open and the return valve open moves the cleaning sleeve to its start position.

### BRIEF DESCRIPTION OF THE DRAWINGS

Embodiments of the disclosure will now be described, by way of example only, with reference to the accompanying drawings, in which:

FIG. 1 shows a compressed and part-sectioned side plan of the UV disinfector with its ancillary components and without the chaotic mixer.

FIG. 2 shows a compressed and part-sectioned side plan of the chaotic mixer.

FIG. 3 shows a compressed and part-sectioned side plan of the UV disinfector with the chaotic mixer fitted in position.

FIG. 4 shows a compressed and part-sectioned plan of the first segment with its ancillary components.

FIG. 5 shows a sectioned side view of the cleaner.

FIG. 6 shows a plan view of a mixing disc.

### DETAILED DESCRIPTION OF THE DRAWINGS

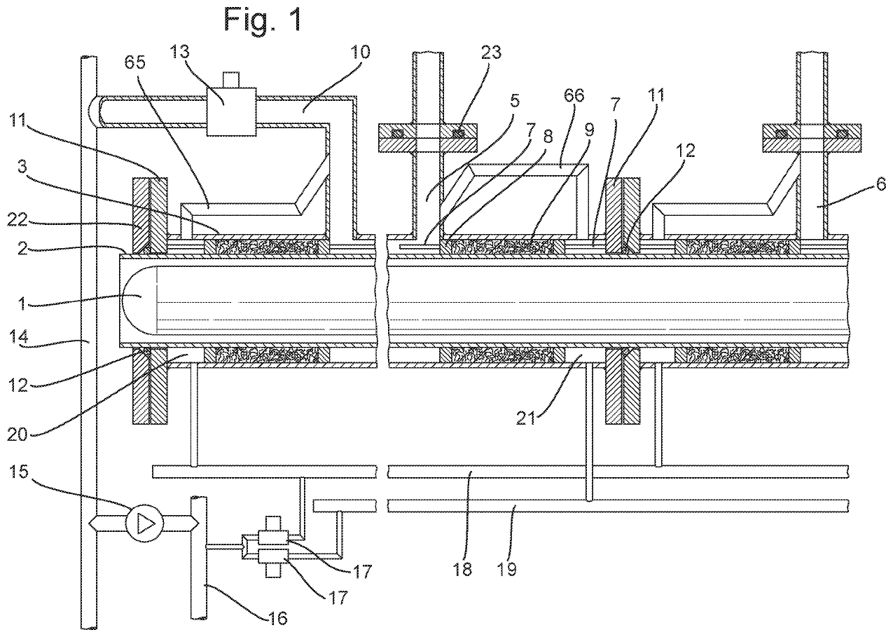

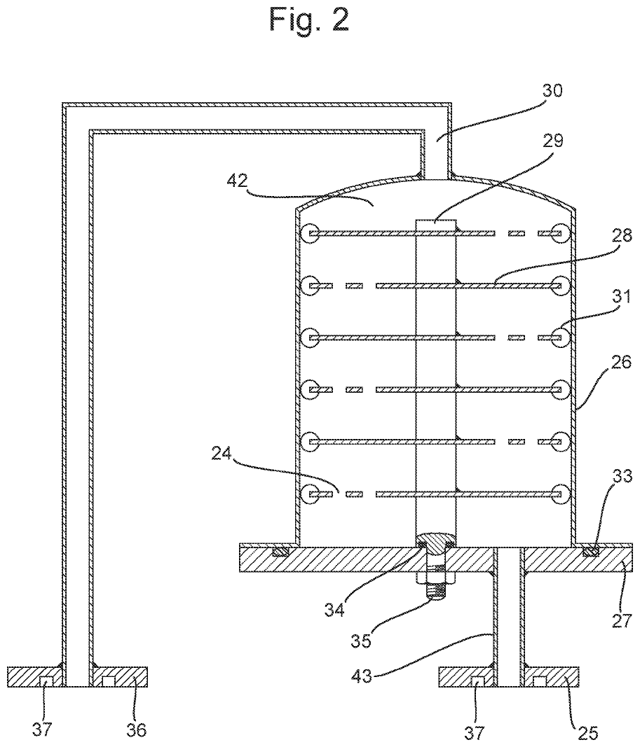

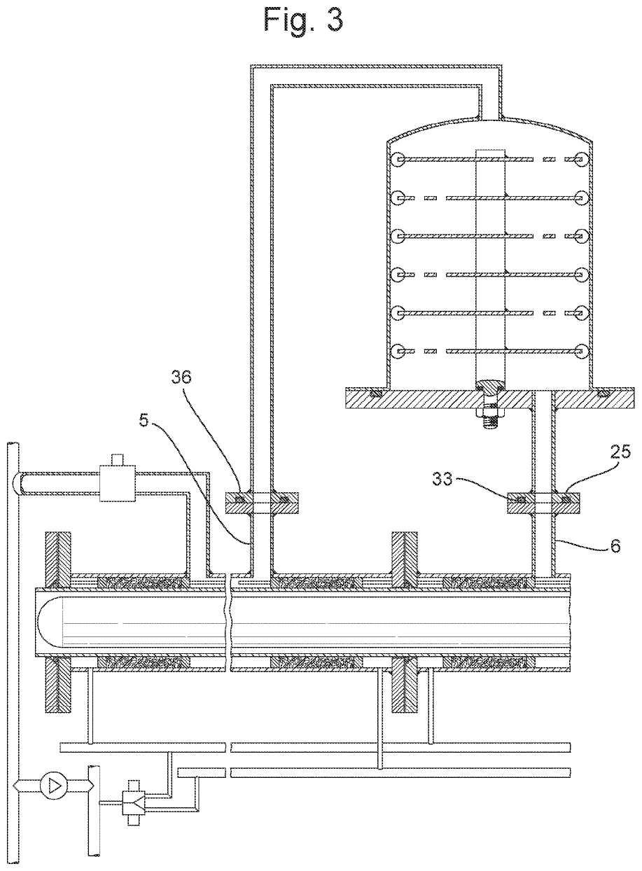

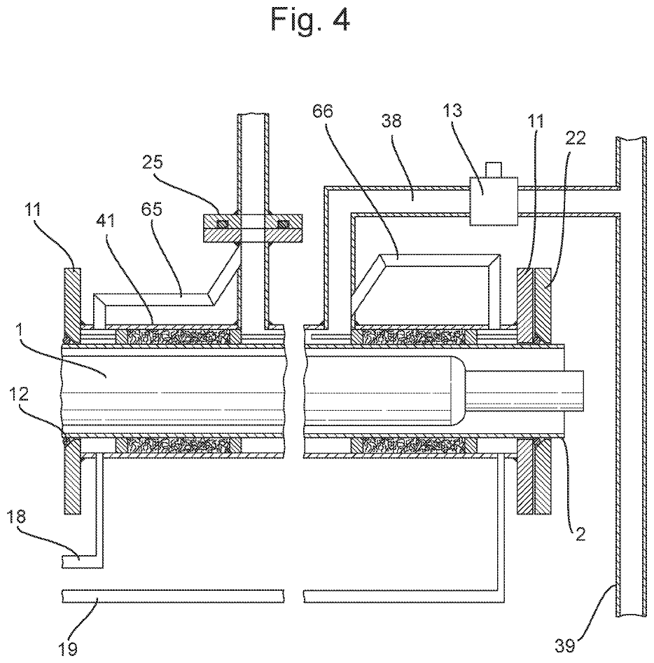

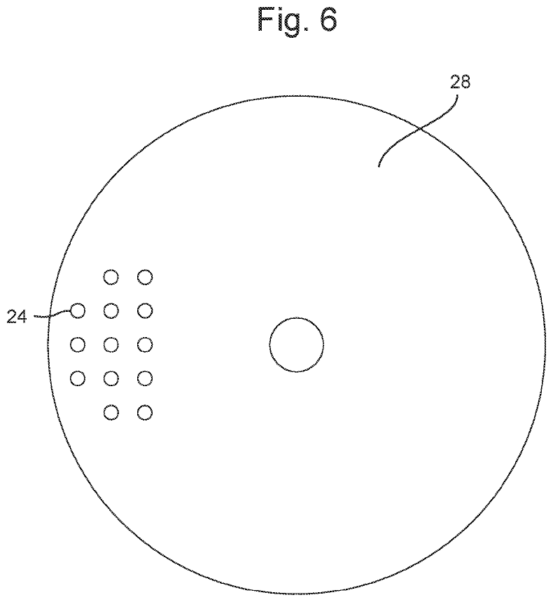

An embodiment of the disclosure will now be described in detail with the aid of FIGS. 1, 2, 3, 4, 5 and 6.

A UV lamp 1 is surrounded centrally by a quartz tube 2 which is in turn surrounded centrally by a metal tube 3. Rigidly fixed to each end of the outside tube are flanges 11. The flanges 11 have a clearance hole to allow the quartz tube 2 to protrude through the flanges 11 and are positioned in the metal tube 3 so that they are central with the metal tube 3.

The UV lamp may be configured to provide UV light having a wavelength in the range of 250 nm to 270 nm. The UV lamp may comprise at least one light emitting diode (LED) configured to provide UV light. The at least one LED may have a power output in the range of 100 W to 1000 W, which is particularly suitable for achieving commercial liquid flows for disinfecting optically dense liquids.

Preferably the metal tube 3 is made from stainless steel.

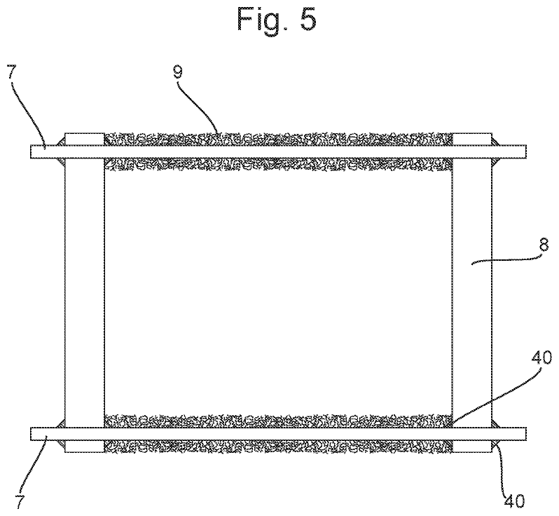

Positioned centrally around the quartz tube 2 inside the metal tube 3 is a cleaning sleeve 9 consisting of two end flanges 8 interposed with a knitted, compressed wire sleeve 9 or spirally wound PTF brush (not shown). The cleaning sleeve may comprise star washers 40, as shown in FIG. 5.

Preferably the end flanges are made of PTFE.

Preferably the end flanges are a small clearance fit between the outside diameter of the quartz tube 2 and the inside diameter of the metal tube 3.

Preferably the cleaning sleeve 9 is made from stainless steel wire.

Preferably the stainless steel wire is coated with virgin PTFE.

An inner diameter of the metal tube 3 may be approximately 5 mm greater than an outer diameter of the quartz tube 2. In other words, the radial gap between the metal tube 3 and the quartz tube 2 is approximately 5 mm.

The interference fit of the cleaning sleeve 9 acts like a circumferential tubular spring. When this cleaning sleeve 9 is moved over the quartz tube 2 it scrubs clean the outside diameter of the quartz tube 2 and the inside surface of the metal tube 3. Attached to one end of the cleaning sleeve 9 is a pin 7 which acts as a stop which positions the cleaning sleeve 9 at its start point in the cleaning cycle. Attached to the other end of the cleaning sleeve 9 is another pin 7 which acts as a stop which positions the cleaning sleeve 9 at its finish point in the cleaning cycle. This subassembly constitutes one segment 41. The segment 41 has an inlet 38 and an outlet 6 positioned such that the cleaning sleeve 9 at the extremities of its travel does not impinge or block either the inlet 38 or the outlet 6.

Means are provided to ensure that the cleaning sleeve always reaches the extremities of its travel in the form of bleed pipe 65 at the start position and bleed pipe 66 at the stop position. These pipes provide conduits so that liquid in the spaces 20 and 21 behind and in front of the cleaning sleeve 9 can escape and not impede the final positioning of the cleaning sleeve 9. The output of the bleed pipes 65 and 66 are positioned in the inlet and outlet of each segment so they create a venturi suction effect to assist the evacuation of the spaces 20 and 21.

Several segments 41 can be clamped together to form a UV disinfector with both the quartz tube 2 and the UV lamp 1 positioned circumferentially central through the whole of the UV disinfector.

Preferably there are at least two segments clamped together.

Preferably there is a plurality of segments clamped together.

The outlet 10 of the last segment and the inlet 38 of the first segment are each fitted with a flow through on/off valve 13 which feeds into the UV disinfector's output manifold 14 and the UV disinfector's inlet manifold 39 so that the UV disinfector's input flow and output flow can be isolated when the on/off valves 13 are switched off.

The segments 41 are made watertight with an “O” ring 12 which when the segments 41 are clamped together compresses the “O” ring 12 between the outside surface of the quartz tube 2 and the flange 11. The first and last segments have clamping rings 22 fitted to form the “O” ring 12 seal.

Straddling the watertight joints of segments 41 are mixer assemblies 42 to mix the fluid; any fluid flowing through the UV disinfector is forced to go through the mixer 42 therefore avoiding any chance of any part of the fluid bypassing the mixing process and therefore ensuring that all of the fluid is mixed.

The mixer comprises a cylindrical body 26 which is domed at one end and flanged at the other end. The cylindrical body 26 is clamped to a base 27 which has a machined “O” ring groove, an “O” ring 33 and clamping holes so when the cylindrical body 26 is clamped to the base 27 it forms a watertight joint. The base 27 has a rigidly fixed inlet pipe 43 so that fluid can enter the cylindrical body 26.

Centrally attached to the base is the mixing apparatus complete with “O” ring 34 which when clamped to the base 27 with stud 35 ensures a watertight seal between the base and the mixing apparatus. The mixing apparatus is designed to produce chaotic mixing of all of the fluid.

The mixing apparatus consists of a central support shaft 29. Means are provided to fix the support shaft to the base in the form of a screw threaded stud 35 at the base of the support shaft. Rigidly attached to the support shaft are mixing discs 28 spaced equally apart. Each disc has a grooved “O” ring 31 attached to its outer rim sized such that when the cylindrical body 26 is attached to the base 27 it provides a watertight seal between the disc 28 and the inside surface of the cylindrical body 26. The discs 28 are thin metal plates with an array of holes 24 at one side of each disc 28 and a central mounting hole to accommodate the support shaft 29. The discs 28 are rigidly fixed to the support shaft 29; the first disc 28 is attached with the array of holes 24 on the same centre line as the inlet pipe 43 but at the opposite side of the disc 28 to the inlet pipe 43. All of the other discs 28 are fixed equally spaced with their array of holes placed at the opposite side to the previous disc and in line with the previous discs array of holes.

When a fluid under pressure enters the mixer 42 from the inlet pipe 43 it immediately hits the base of the first disc 28 where the water molecules scatter in a random pattern providing chaotic mixing. Due to the pressure the fluid is forced to move through the array of holes 24 at the other end of the disc producing jets of fluid which hit the base of the second disc 28 and creates further chaotic mixing. This effect carries on as the fluid progresses through the mixer 42.

Means are provided to deliver the mixed fluid out of the mixer 42 and into the UV disinfector in the form of an outlet pipe 30 rigidly fixed to the centre of the domed top of the cylindrical body 26. Both the inlet 43 and the outlet 30 have flanges 25, 36 rigidly attached; both flanges have an “O” ring groove 37 so that when the flanges 25, 36 are clamped with an “O” ring 23 onto the outlet 5 and inlet 6 of the segments they form a watertight seal.

Means are provided to move the cleaner 9 from its start position to its stop position and from its stop position to its start position in the form of two flow through on/off valves 17. The forward valve 17 feeds into the forward manifold 18 and the return valve 17 feeds into a return manifold 19. The forward manifold 18 feeds fluid into the space 20 behind the cleaning sleeve 9 when it is in its start position and the return manifold 19 feeds into the space 21 in front the cleaning sleeve 9 when it is in its stop position. Feeding the two valves is the cleaning manifold 16 which in turn is fed from a pump 15 whose inlet is fed from the UV disinfector's outlet manifold 14. The pump 15 draws fluid from the UV disinfector's output manifold 14 and pressurizes the cleaner manifold 16 with a pressure higher than the inlet manifold 39 pressure and with the output on/off valve 13 shut and the valve 17 feeding the forward manifold 18 feeds fluid from the forward manifold 18 into the space 20 behind the cleaning sleeve 9 moving the cleaning sleeve 9 to its stop position at which point the valve 17 is switched off.

With the input and output on/off valves 13 open and the valve 17 feeding the return manifold 19 feeds fluid from the return manifold 19 into the space 21 in front of the cleaning sleeve 9 moving the cleaning sleeve 9 towards its stop position, after a short period of time valve 17 is switched off and the inlet pressure continues to push the cleaning sleeve 9 to its stop position.

Preferably the valve 13 in conjunction with the twin valves 17 are made to switch in a sequence which allows the cleaning sleeve 9 to move in a forward and backwards motion to impart a scrubbing action to the surface of the quartz tube 2.

The UV disinfectors can be connected in series to increase UV dose or parallel to increase the volume of disinfected or a combination of both. When the cleaning cycle is in operation the displaced fluid is pushed back into the inlet manifold 39 and dissipated through the other UV disinfectors.

It will be appreciated from the above description that many features of the different examples are interchangeable and combinable. The disclosure extends to further examples comprising features from different examples combined together in ways not specifically mentioned. Indeed, there are many features presented in the above examples and it will be apparent to the skilled person that these may be advantageously combined with one another.

1. An apparatus for disinfection of optically dense fluid, the apparatus comprising:
a metal tube;
a quartz tube surrounded by the metal tube;
a UV lamp surrounded by the quartz tube;
a chamber defined between the quartz tube and the metal tube, the chamber having an inlet and an outlet;
a cleaning sleeve positioned in the chamber and configured to be movable along the quartz tube between a start position and a stop position; and
a valve arrangement configured to transfer optically dense fluid to spaces behind and in front of the cleaning sleeve to thereby move the cleaning sleeve between its start position and its stop position.

2. The apparatus of claim 1, wherein the UV lamp is configured to provide UV light having a wavelength in the range from 250 nm to 270 nm.

3. The apparatus of claim 1, further comprising a flange rigidly fixed to each end of the metal tube, the flanges comprising a clearance hole to allow the quartz tube to protrude through the flanges so that the flanges are positioned centrally with the metal tube.

4. The apparatus of claim 1, wherein the metal tube is made of stainless steel and an inside surface of the metal tube is coated with polytetrafluoroethylene (PTFE).

5. The apparatus of claim 1, wherein the cleaning sleeve comprises two end flanges interposed by compressed wire sleeve.

6. The apparatus of claim 1, wherein an inner diameter of the metal tube is approximately 5 mm greater than an outer diameter of the quartz tube.

7. The apparatus of claim 1, wherein at least one end of the cleaning sleeve comprises a pin configured to limit the travel of the cleaning sleeve.

8. The apparatus of claim 1, wherein the inlet and the outlet of the chamber are positioned such that fluid communication with the chamber is maintained when the cleaning sleeve is at its start position and its stop position.

9. The apparatus of claim 1, further comprising at least one of:
a bleed pipe configured to provide a conduit between the outlet and the space behind the cleaning sleeve; and
a bleed pipe configured to provide a conduit between the inlet and the space in front of the cleaning sleeve.

10. The apparatus of claim 1, wherein the valve arrangement comprises:
a forward valve configured to feed optically dense fluid into the space behind the cleaning sleeve via a forward manifold;
a return valve configured to feed optically dense fluid into the space in front of the cleaning sleeve via a return manifold; and
a pump configured to pump optically dense fluid from the outlet of the chamber to the forward valve and the return valve.

11. The apparatus of claim 1, further comprising a mixer for chaotically mixing the optically dense fluid between the inlet and the outlet.

12. The apparatus of claim 1, wherein the cleaning sleeve is a first cleaning sleeve and wherein the apparatus further comprises a second cleaning sleeve, the second cleaning sleeve positioned in the chamber and configured to be movable along the quartz tube between a start position and a stop position.

13. The apparatus of claim 12, wherein the first cleaning sleeve is disposed in a first chamber segment and the second cleaning sleeve is disposed in a second chamber segment fluidically separated from the first chamber segment by a flange.

14. The apparatus of claim 12, wherein the valve arrangement is further configured to transfer optically dense fluid to spaces behind and in front of the second cleaning sleeve to thereby move the second cleaning sleeve between its start position and its stop position.

15. A mixer for mixing fluid, the mixer comprising:
a cylindrical body;
an inlet pipe for transporting fluid into the cylindrical body;
an outlet pipe for transporting fluid out of the cylindrical body;
a central support shaft; and
a plurality of mixing discs rigidly attached to the central support shaft via a central mounting hole;
wherein each of the plurality of mixing discs comprises an array of holes on one side of the mixing disc; and
wherein the mixing discs are arranged such that pressurised fluid entering the inlet pipe is forced through the array of holes of each of the plurality of mixing discs to provide chaotic mixing of the fluid before reaching the outlet pipe.

16. The mixer of claim 15, wherein the plurality of mixing discs comprises:
a first mixing disc comprising a first array of holes; and
a second mixing disc, adjacent to the first mixing disc, and comprising a second array of holes;
wherein the first array of holes is on the opposite side to the second array of holes.

17. The mixer of claim 15, wherein the inlet pipe is provided at a base of the cylindrical body and is offset from the central support shaft by a first radial distance.

18. The mixer of claim 17, wherein at least one of the plurality of mixing discs is arranged such that its array of holes is provided at the first radial distance.

19. A method for disinfection of optically dense fluid, the method comprising:
directing fluid to a chamber, the chamber being defined between a metal tube and a quartz tube that surrounds a UV lamp;
irradiating the fluid with UV light from the UV lamp;
directing the fluid from the chamber to a valve arrangement, the valve arrangement configured to direct fluid to spaces behind and in front of a cleaning sleeve surrounding the quartz tube in the chamber; and
scrubbing the quartz tube with the cleaning sleeve, wherein scrubbing comprises:
opening a first valve of the valve arrangement to direct fluid to the space behind the cleaning sleeve to move the cleaning sleeve forwards; and
closing the first valve and opening a second valve of the valve arrangement to direct fluid to the space in front of the cleaning sleeve to move the cleaning sleeve backwards.

20. The method of claim 19, wherein the step of directing fluid to the space behind or in front of the cleaning sleeve is performed at a higher pressure than the step of directing fluid to the chamber.
